(19) United States
(12) Patent Application Publication
(10) Pub. No.: US 2024/0050780 A1
(43) Pub. Date: Feb. 15, 2024

(54) HEAD WEARABLE AIR PURIFIER

(71) Applicant: Dyson Technology Limited, Wiltshire (GB)

(72) Inventor: Nikian Naji AGHABABAIE, London (GB)

(73) Assignee: Dyson Technology Limited, Wiltshire (GB)

(21) Appl. No.: 18/267,741

(22) PCT Filed: Oct. 7, 2021
(86) PCT No.: PCT/GB2021/052597

§ 371 Date: Jun. 15, 2023

(30) Foreign Application Priority Data
Dec. 18, 2020 (GB) 2020104.2

(51) Int. Cl. A62B 18/00; A62B 23/00; A61L 2/08

(52) U.S. Cl. CPC A62B 18/003; A62B 23/00; A61L 2/084; A62B 18/006; A61L 2209/14

(57) ABSTRACT

A head wearable air purifier that includes a filter assembly, a motor-driven impeller for creating an airflow through the filter assembly to obtain a filtered airflow downstream of the filter assembly, and at least one light source for emitting light in a violet portion of the visual spectrum. The head wearable air purifier further includes a light guide arranged to guide light emitted from the at least one light source to illuminate at least part of an upstream surface of the filter assembly for the decontamination thereof.

### FIELD OF THE INVENTION

The present invention relates to a head wearable air purifier. In particular, the invention relates to the use of light in a violet portion of the visual spectrum to decontaminate at least part of a filter assembly of the head wearable air purifier.

### BACKGROUND

Air pollution is an increasing problem and a variety of air pollutants have known or suspected harmful effects on human health. The adverse effects that can be caused by air pollution depend upon the pollutant type and concentration, and the length of time that a person is exposed to the polluted air. For instance, relatively high air pollution levels can cause immediate health problems such as aggravated cardiovascular and respiratory illness, whereas long-term exposure to polluted air can have permanent health effects such as loss of lung capacity and decreased lung function, and the development of diseases such as asthma, bronchitis, emphysema, and possibly cancer. Airborne pathogens are also an issue that can cause various health risks when individuals are exposed to (i.e. breathe in) such pathogens.

In locations with particularly high levels of air pollution, many individuals have recognised the benefits of minimising their exposure to these pollutants and have taken to wearing face masks with the aim of filtering out at least a portion of the pollutants present in the air before it reaches the mouth and nose. These face masks range from basic dust masks that merely filter out relatively large dust particles, to more complex air-purifying respirators that the air pass through a filter element or cartridge. However, as these face masks typically cover at least a user's mouth and nose they can make normal breathing more laborious and can also make it difficult for the user to speak effectively to others, such that there is some reluctance to make use of such face masks on a day-to-day basis despite the potential benefits.

As a consequence, there have been attempts to develop air purifiers that can be worn by the user but that do not require the user's mouth and nose to be covered. For instance, one design for a head wearable air purifier includes two ear assemblies to be worn over respective ears of a user and connected by a headband, similar to a typical pair of headphones. Each ear assembly may include a motor-driven impeller and a filter assembly, where the motor-driven impeller creates an airflow through the filter assembly to obtain a filtered airflow downstream thereof. The filtered airflow may then be directed to a user's mouth. For instance, in one design the head wearable air purifier includes a nozzle or visor that is connected at each end to the respective ear assemblies and, in use, is positioned next to the user's mouth and nose. The nozzle may receive the filtered air downstream of the filter assembly and direct the air to an opening or nozzle output that is positioned adjacent to the user's mouth and nose in use so as to deliver filtered air to the user.

An issue with such an air purifier is that air drawn into the filter assembly may be obtained from an environment that may contain many different kinds of contamination. Larger dirt and dust particles may be filtered out, but smaller contaminants such as bacteria and other microbes are drawn in too. Such microbial contamination may then gather and grow in and around the filter assembly. Cleaning the hard surfaces of an air purifier may be performed using a wet cloth, for instance. However, this can actually lead to an increase in levels of microbial contamination. In any case, it may be difficult, or not possible, to access certain parts of a filter assembly to be decontaminated with such a cloth particularly without damaging the filtration surfaces. A further constraint when considering how the filter assembly of such air purifiers may be cleaned is that there is generally a relatively small amount of space in the vicinity of the filter assembly in which physical devices to aid decontamination may be fitted or positioned.

It is against this background to which the present invention is set.

### SUMMARY OF THE INVENTION

According to an aspect of the present invention there is provided a head wearable air purifier, comprising a filter assembly, a motor-driven impeller for creating an airflow through the filter assembly to obtain a filtered airflow downstream of the filter assembly, and at least one light source for emitting light in a violet portion of the visual spectrum. The head wearable air purifier comprises a light guide arranged to guide light emitted from the at least one light source to illuminate at least part of an upstream surface of the filter assembly for the decontamination thereof.

The light guide may comprise light piping means arranged to guide light emitted from the at least one light source through the light piping means to the upstream surface of the filter assembly.

The light piping means may comprise at least one corner arranged to cause a change of direction of light being guided through the light piping means.

At least part of the light piping means may extend through at least one structural component of the head wearable air purifier.

The at least one structural component may include a filter frame that is arranged to support the filter assembly.

The light guide may be arranged to emit light in a direction substantially parallel to the upstream surface of the filter assembly.

The light guide may be arranged to emit light in a plurality of directions along the upstream surface of the filter assembly.

The head wearable air purifier may comprise a filter shroud for covering the upstream surface of the filter assembly. Light from the at least one light source may illuminate a gap defined between the filter shroud and the upstream surface of the filter assembly.

An inner surface of the filter shroud, adjacent to the upstream surface of the filter assembly, may comprise, or be made from, a light reflective coating.

A part of the light guide from which light, from the at least one light source, is emitted may be positioned between the filter shroud and the upstream surface of the filter assembly.

The filter shroud may comprise a lip portion for fixing the filter shroud in place to cover the upstream surface of the filter assembly.

At least part of the light guide may be attached to the filter shroud.

The at least one light source may be arranged at a side of the filter assembly opposite to the upstream surface of the filter assembly.

The at least one light source may be attached to, or disposed adjacent to, at least one structural component of the head wearable air purifier.

The at least one structural component may be between the at least one light source and the light guide.

The at least one structural component may be formed from a material that allows light emitted from the at least one light source to pass therethrough.

The material may be a plastic material. Optionally, the plastic material may be acrylic, polycarbonate, polypropylene or include the use of polylactic acid.

The at least one structural component may include a filter frame that is arranged to support the filter assembly.

The at least one light source may be in the form of one or more LEDs.

The at least one light source may be configured for emitting light with a wavelength of about 405 nm.

The head wearable air purifier may comprise switch means for switching the at least one light source between an on state, in which the light source emits light, and an off state, in which the light source does not emit light. Optionally, the switch means may be user operable.

The head wearable air purifier may comprise an ear assembly arranged to be worn over an ear of a user, wherein the ear assembly includes the filter assembly, the motor-driven impeller and the at least one light source. Optionally, the ear assembly may be a first ear assembly arranged to be worn over a first ear of the user. The head wearable air purifier may comprise a second ear assembly arranged to be worn over a second ear of a user, wherein the second ear assembly may include a second filter assembly, a second motor-driven impeller and at least one second light source, and wherein the second filter assembly, second motor-driven impeller and at least one second light source may be according to the filter assembly, the motor-driven impeller and the at least one light source, respectively, as described above.

The head wearable air purifier may include a nozzle for receiving the filtered air downstream of the filter assembly, and for directing the filtered air towards a user's mouth and nose.

### BRIEF DESCRIPTION OF THE DRAWINGS

Examples of the invention will now be described with reference to the accompanying drawings, in which:

FIG. 1 shows a schematic view of a head wearable air purifier according to an example of the invention;

FIG. 2 shows a cross section of an ear assembly of the head wearable air purifier of FIG. 1;

FIG. 3 shows a top view of an outer cover of the ear assembly of FIG. 2;

FIG. 4 shows a nozzle of the head wearable air purifier of FIG. 1;

FIG. 5 shows part of the cross section of the ear assembly of FIG. 2;

FIG. 6 shows the cross section of the ear assembly of FIG. 2 with an outer cover of the ear assembly removed; and,

FIGS. 7(a) and 7(b) show top and bottom perspective views of a filter frame of the ear assembly of FIG. 2; and,

FIGS. 8(a) and 8(b) show bottom views of the outer cover of FIG. 3.

### DETAILED DESCRIPTION

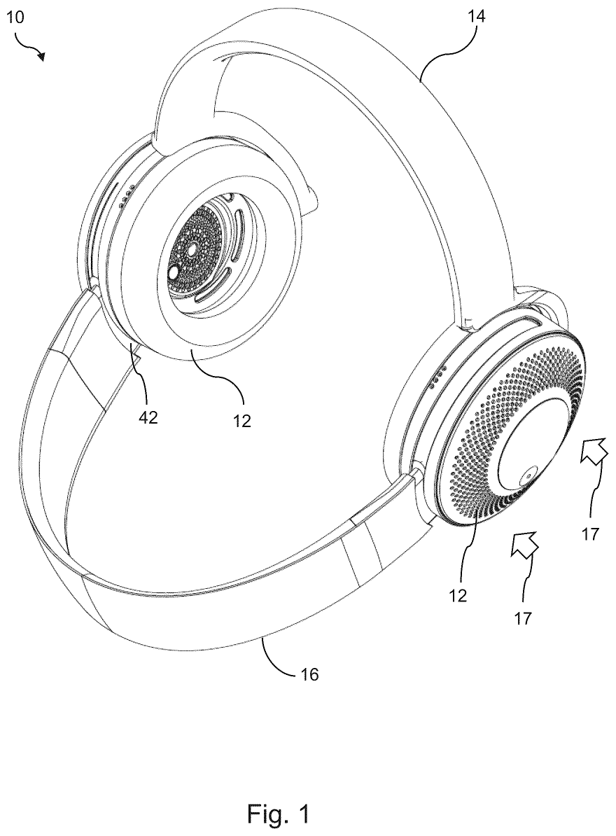

FIG. 1 is an external view of an example of a head wearable air purifier 10. The head wearable air purifier 10 includes a pair of generally cylindrical ear assemblies or cups 12 connected to an arcuate headband 14, and a nozzle or visor 16 that that extends between, and is connected at, opposite ends to the ear assemblies 12. Air to be filtered is drawn into the ear assemblies 12 in a direction generally indicated by the arrows 17, as will be discussed in greater detail below.

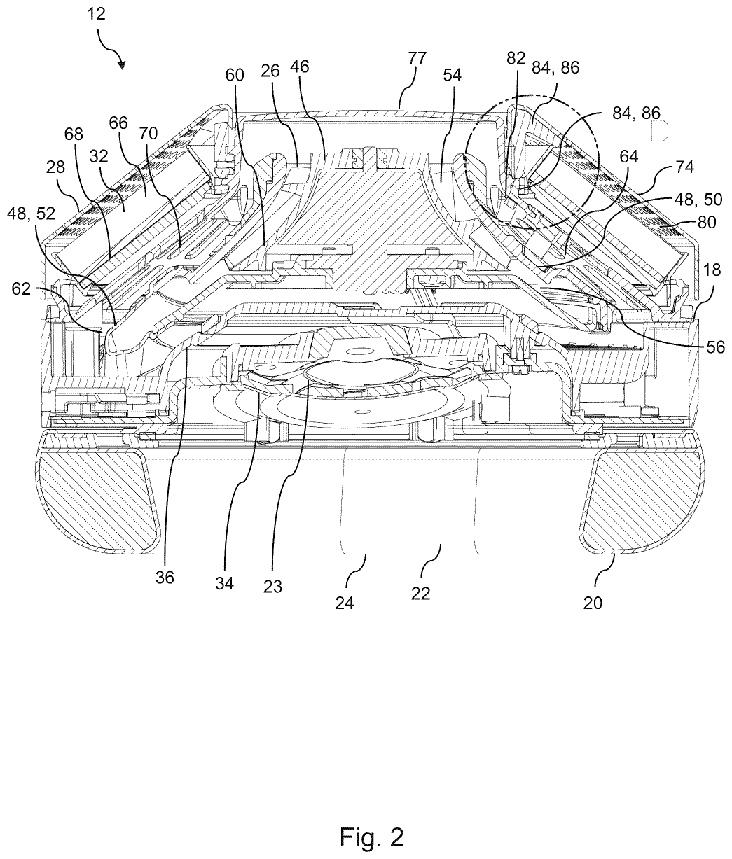

With additional reference to FIG. 2, each of the ear assemblies 12 includes a housing 18 and an ear pad 20 attached to the housing 18, with the housing 18 and ear pad together defining a cavity 22 having an opening 24. An optional speaker or acoustic driver unit 23 may be attached to the housing 18 such that it is at least partially exposed to the cavity 22.

Each of the ear assemblies 12 includes a motor-driven impeller 26 disposed within the housing that is arranged to create an airflow through the housing 18. The housing 18 is therefore provided with an air inlet 28 through which an airflow can be drawn into the housing 18 by the motor-driven impeller 26, and an air outlet for emitting the airflow from the housing 18.

A filter assembly 32 is also disposed within the housing 18 such that the airflow generated by the motor-driven impeller 26 passes through the filter assembly 32 and such that the airflow emitted from the ear assembly 12 is filtered/purified by the filter assembly 32. The filter assembly 32 is therefore located downstream (relative to the airflow generated by the motor-driven impeller 26) of the air inlet 28 of the housing 18 and upstream of the air outlet. In the described example, the filter assembly 32 is also located upstream of, or relative to, the motor-driven impeller 26.

In the described example, the housing 18 includes a speaker chassis 34 upon which the optional acoustic driver unit 23 sits, and a generally frustoconical speaker cover 36 mounted on the speaker chassis 34 over the acoustic driver unit 23. The speaker chassis 34 includes a generally circular base that is surrounded by a cylindrical side wall. The air outlet of the housing 18 is then defined by an aperture formed in the cylindrical side wall. The ear assembly 12 is also provided with a hollow, rigid outlet duct 42 that extends from the housing 18 and that is arranged to connect the air outlet 30 of the ear assembly 12 to an air inlet of the nozzle 16. The rigid outlet duct 42 can in some examples be regarded as being part of the housing 18. In different examples, one or both of the ear assemblies may not include an acoustic driver unit (or the associated components) such that the head wearable air purifier may not additionally operate as headphones.

Each of the ear assemblies 12 also includes one or more circuit boards upon which various circuitry is disposed or mounted. For instance, this electronic circuitry may include motor control circuitry that is arranged to control a rotational speed of a motor 46 that drives the impeller 26. In the described example, the circuit board(s) is disposed on, or mounted to, the peripheral portion of the speaker chassis 34. The circuit board may therefore at least partially encircle the acoustic driver unit 23 (if included).

A generally frustoconical impeller casing 48 containing both the impeller 26 and the motor 46 may be disposed over the speaker cover 36 (if included). In some examples, the impeller casing 48 may be regarded as being part of the housing 18 of the ear assembly 12. This impeller casing 48 includes a generally frustoconical impeller housing 50 surrounding the impeller 26 and the motor 46, and an annular volute 52 fluidically connected to a base of the impeller housing 50, where the annular volute 52 is arranged to receive air exhausted from the impeller housing 50. The impeller housing 50 is provided with an air inlet 54 through which air can be drawn by the impeller 26, and an air outlet 56 through which the air is emitted from the impeller housing 50 to the annular volute 52. The air inlet 54 of the impeller housing 50 is provided by an aperture/opening at the small diameter end of the impeller housing 50 and the air outlet 56 is provided by an annular slot formed around a large diameter end of the impeller housing 50.

The annular volute 52 includes a spiral (i.e. gradually widening) duct that is arranged to receive the air exhausted from the impeller housing 50 and to guide the air to an air outlet of the volute 52. The air outlet of the volute 52 is fluidically connected to the air outlet of the ear assembly 12. The term ‘volute’ as used herein refers to a spiral funnel that receives the fluid being pumped by an impeller and increases in area as it approaches a discharge port. The air outlet of the volute 52 therefore provides an efficient and quiet means for collecting the air that is exhausted from the circumferential annular slot that that forms the air outlet 56 of the impeller housing 50.

In the described example, the impeller 26 is a mixed flow impeller that has a generally conical or frustoconical shape. The impeller 26 is hollow such that a rear/back side of the impeller 26 defines a generally frustoconical recess. The motor 46 is then nested/disposed within this recess. The impeller 26 may be a semi-open/semi-closed mixed flow impeller, i.e. having a back shroud 60 only. The back shroud 60 of the impeller 26 then defines the recess within which the motor 46 is nested/disposed.

The impeller casing 48 may optionally be supported/suspended within the housing 18 by a plurality of resilient supports 62 that reduce the transmission of vibrations from the impeller casing 48 to the housing 18. To do so, the plurality of resilient supports 62 may each comprise a resilient material such as an elastomeric or rubber material.

The filter assembly 32 may be mounted to the speaker chassis 34 so that the filter assembly 32 is provided upstream of the impeller 26 and is arranged to be nested over the impeller casing 48. A filter seat or frame 64 supports the filter assembly 32, which has one or more filter elements 66, 68. In the described example, the filter assembly 32 includes both a particulate filter element 66 and a chemical filter element 68, with the particulate filter element 66 located upstream relative to the chemical filter element 68. In different examples, the filter assembly may include only one of these filter elements, or may include different filter elements, optionally in combination with either one or both of the filter elements 66, 68 of the described example.

The filter seat 64 is provided with a plurality of apertures 70 that allow air to pass from a front surface of the filter seat 64 to a rear/back surface of the filter seat 64, with the front surface being arranged to support the filter elements 66, 68 over the plurality of apertures 70. The filter seat 64 then further defines an air passageway or channel 72 between the rear/back surface of the filter seat 64 and the air inlet 54 of the impeller casing 48 that is arranged to guide air to the air inlet 54 of the impeller casing 48. This air passageway 72 is provided by a cavity defined between the rear/back surface of the filter seat 64 and a front surface of the impeller casing 48. Air must therefore pass through the filter elements 66, 68 before it can pass through the apertures 70 in the filter seat 64 and into the air passageway that leads to the air inlet 54 of the impeller casing 48.

In the described example, the filter seat 64 is mounted to the speaker chassis 34 and located over the impeller housing 50, with the impeller housing 50 partially disposed within a volume defined by a back of the filter seat 64. In particular, the filter seat 64 may include a generally frustoconical peripheral portion and a generally cylindrical central portion. The generally frustoconical peripheral portion of the filter seat 64 is provided with the plurality of apertures 70 and is arranged to support one or more generally frustoconical filter elements 66, 68 over the plurality of apertures 70. The impeller housing 50 may be at least partially disposed within the generally cylindrical central portion of the filter seat 64. In particular, the air inlet 54 of impeller housing 50 may be disposed within a volume defined by a back of the cylindrical central portion of the filter seat 64.

Structural components or parts of the ear assembly 12, such as the housing 18, impeller casing 48 and filter frame 64, may together be regarded as a body of the ear assembly. One or more of the structural components may be formed from a plastics material, e.g. acrylic. In particular, the filter frame 64 may be formed, at least in part, from acrylic.

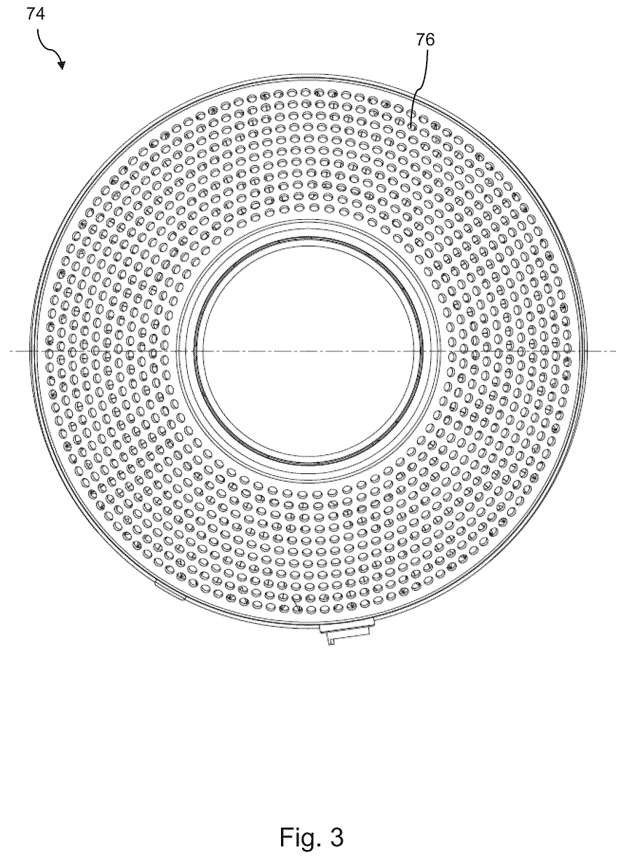

With additional reference to FIG. 3, the (body of the) ear assembly 12 further includes an outer cover 74 that may be mounted onto the speaker chassis 34. This outer cover 74 is arranged to fit over the filter assembly 32, and therefore the outer cover 74 generally conforms to the filter assembly 32. The outer cover 74 is provided with an array of apertures 76 that allow air to pass through the outer cover 74, and so the apertures define an air inlet of the outer cover 74. These apertures are sized to prevent larger particles from passing through to the filter assembly 32 and blocking, or otherwise damaging, the filter elements 66, 68. Alternatively, in order to allow air to pass through, the outer cover 74 could comprise one or more grilles or meshes mounted within windows in the outer cover 74. It will also be clear that alternative patterns of arrays are envisaged within the scope of the present disclosure.

The outer cover 74 is releasably attached to the speaker chassis 34 so as to cover the filter assembly 32. For instance, the outer cover 74 could be attached to the speaker chassis 34 using a catch mechanism. When mounted on the speaker chassis 34, the outer cover 74 protects the filter elements 66, 68 from damage, for example during transit, and also provides a visually appealing outer surface covering the filter assembly 32, which is in keeping with the overall appearance of the purifier 10.

In the described example, the outer cover 74 is provided as a hollow frusta-cone with open ends. The open large diameter end of the outer cover 74 may be arranged to fit over the periphery of the large diameter end of the filter assembly 32, whilst the open small diameter end of the outer cover 74 may be arranged fit over both the periphery of the small diameter end of the filter assembly 32 and the generally cylindrical central portion of the filter frame 64. A circular front surface 77 of the generally cylindrical central portion of the filter frame 64 may therefore be exposed within the open small diameter end of the outer cover 74 and may thereby form a portion of the outer surface of the ear assembly 12. The circular front surface 77 of the filter frame 64 may be transparent such that it forms a window through which the user can see the spinning of the impeller 26 through the air inlet 54 of the impeller casing 48. This would allow the user to visually check the speed of the impeller 26 and to confirm that the impeller 26 is functioning appropriately.

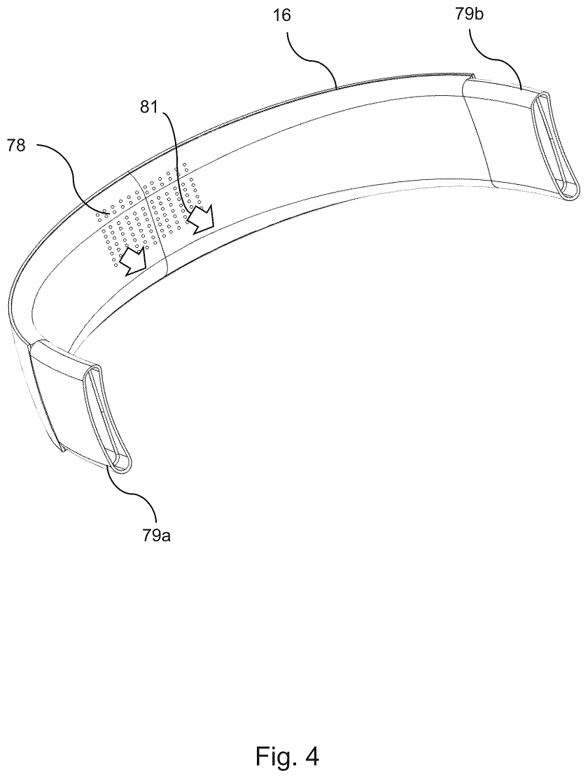

Returning to FIG. 1, and with additional reference to FIG. 4, in the described example a first open end 79a of the nozzle 16 is connected to the rigid outlet duct 42 that extends from the housing 18 of a first one of the ear assemblies 12. The nozzle 16 extends away from the first ear assembly 12 and may assume an arcuate shape so that the opposite, second open end 79b of the nozzle 16 connects to the rigid outlet duct 42 that extends from the housing 18 of a second one of the ear assemblies 12. The nozzle 16 may be arranged such that, when the purifier 10 is worn by a user with the first ear assembly 12 over a first ear of the user and the second ear assembly 12 over a second ear of the user, the nozzle 16 can extend around the user's face, from one side to the other, and in front of (i.e. adjacent to) a mouth of the user. In particular, the nozzle 16 may extend around the jaw of the user, from adjacent to one cheek to adjacent the other cheek, without contacting the mouth, nose or surrounding regions of the user's face. It may therefore be preferable that at least a portion of the nozzle 16 is formed of a transparent or partially transparent material so that the user's mouth is visible through the nozzle 16 so as to avoid limiting the user's ability to speak to others in a clear manner. For instance, a central portion of the nozzle 16 could be made from a flexible, transparent plastic such as a polyurethane, whilst the two end portions could each made from a stiff, transparent plastic such as a polyethylene terephthalate glycol-modified (PETG). Alternatively, the entire nozzle 16 could be formed from a single transparent or partially transparent material.

The nozzle 16 is provided with an air outlet 78 for emitting/delivering the filtered air to a user. For instance, the air outlet 78 of the nozzle 16 can comprise an array of apertures formed in a section of the nozzle 16, with these apertures extending from an interior passage defined by the nozzle 16 to an exterior surface of the nozzle 16. Alternatively, the air outlet 78 of the nozzle 16 may comprise one or more grilles or meshes mounted within windows in the nozzle 16.

In use, the purifier 10 is worn by a user with the first ear assembly 12 over a first ear of the user and the second ear assembly 12 over a second ear of the user such that the nozzle 16 can extend around a face of the user, from one ear to the other, and over at least the mouth of the user. Within each ear assembly 12, the rotation of the impeller 26 by the motor 46 will cause an airflow to be generated through the impeller casing 48 that draws air into the ear assembly 12 through the apertures in the outer cover 74. This flow of air will then pass through the filter elements 66, 68 disposed between the outer cover 74 and the filter seat 64, thereby filtering and/or purifying the airflow. The resulting filtered airflow will then pass through the apertures 70 provided in the frustoconical portion of the filter seat 64 into the air passageway 72 provided by the space between the impeller casing 48 and the opposing surface of the filter seat 64, with the air passageway 72 then guiding the airflow to the air inlet 54 of the impeller casing 48. The impeller 26 will then force the filtered airflow out through the annular slot that provides the air outlet 56 of the impeller housing 50 and into the volute 52 of the impeller casing 48. The volute 52 then guides the filtered airflow through the air outlet of the ear assembly 12, through the rigid outlet duct 42 that extends from the housing 18, into the nozzle 16 through an air inlet provided by one of the open ends 79a, 79b of the nozzle 16, and out through the air outlet 78 of the nozzle 16 in a direction generally indicated by the arrows 81 (towards a user's mouth in use).

An issue with a head wearable air purifier as described above is that the filter assembly will become contaminated or clogged with bacteria or other particles from the air as it removes these from air passing therethrough during use of the purifier. The filter assembly therefore needs to be cleaned to remove such particles. Otherwise, the effectiveness of the filter assembly will reduce over time, both in terms of the effectiveness of the filters at removing pollutants from the air and the amount of air that can be driven through the filter assembly which can then be provided to a user. One way in which the filter assembly could be cleaned would be to wipe down its surfaces; however, this would be disadvantageous in that a cloth that is used for this may actually already include microbes that are then disposed onto the filter assembly, and also that it can be difficult to access the parts of a filter assembly to be cleaned in this way.

One way in which surfaces are decontaminated is by treating the surfaces with light. In particular, light of certain wavelengths is known to be very effective at killing any microbes that may have accumulated on the surfaces that are then illuminated by such light. Specifically, light in the violet portion of the visual spectrum is known to be effective for this purpose. The violet portion of the visual spectrum is typically defined as spanning the range of about 380-450 nm. The light used may therefore have a wavelength of about 405 nm, for instance. The use of violet visible light for this particular implementation brings a number of advantages that are not found in UV or near UV light. For example, the low energy visible light does not damage the material of the surfaces it illuminates. This is especially advantageous because many user appliances or gadgets are at least partially made of plastics that are easily damaged by UV light. Another important advantage of violet visible light is that no direct line of sight between the light source and the surface or part to be cleaned is needed. Indirect irradiation of violet visible light helps to get rid of the microbial contamination too.

Some filter media can include antimicrobial coatings or additives within the fibres which would aid in keeping the filter sterile. However, over the use of the filter, particles and pathogens will cover the fibres prohibiting the contact of subsequent microbes being inactivated on the surface of said fibres. Hence, the use of light to continuously inactivate pathogens throughout the life of the filter's use provides improved cleanliness. The use of visible light to enforce such an effect as opposed to ultraviolet wavelengths also has the benefit of improved safety being in such close proximity to the wearer and other people close by. As a result, fewer safety preventative measures are required for equivalent efficacy.

It is to be noted that emitting light in a violet portion of the visual spectrum as part of a decontamination process means that the emitted light contains a significant portion of light in that part of the electromagnetic spectrum and that the intensity of that significant portion is sufficient to have a useful anti-microbial and decontaminating effect. The emitted light does not need to be exclusively in the violet portion of the visual spectrum. Provided that there is a sufficient intensity of light in that portion of the spectrum, and preferably at or around the 405 nm wavelength, for achieving a decontaminating effect, light from other parts of the electromagnetic spectrum may also be emitted. Further, it is noted that as part of the decontamination process the intensity of the emitted light may vary over time. Such variations may be gradual and continuous, or in the form of a pattern of light pulses. If pulsed light is used, the frequency, duration and intensity of the pulses may either be constant or varying.

It has been recognised that an upstream surface of a filter assembly—i.e. the side through which air is drawn into the filter assembly—of a head wearable air purifier as described above is particularly susceptible to becoming contaminated or clogged with particles, microbes or bacteria. It is therefore crucial that this upstream side of a filter assembly is decontaminated. As may be seen in FIG. 2, the upstream surface or side 80 of the filter assembly 32 may be regarded as an outwardly-facing surface of the filter assembly 32, opposite to a side of the filter assembly 32 facing a user's ear when the air purifier is in use. If a filter assembly includes more than one filter component, then it may be that the filter component that is further upstream is the one that benefits the greatest from sterilisation.

Light in the violet portion of the visual spectrum may be used to decontaminate the upstream surface 80 (or, more generally, the upstream filter 66) of the filter assembly 32. It would be advantageous for a source of violet light to be provided in, or as part of, the air purifier 10 so that decontamination may be performed when the air purifier 10 is in use and being worn by a user. However, as will be apparent from FIGS. 1 and 2, there are relatively severe space constraints in the ear assemblies 12 of the air purifier 10, and access to the upstream surface 80 of the filter assembly 32 is particularly limited. Indeed, it would be difficult, or impossible, to provide a light source for emitting violet light in the vicinity of the filter assembly upstream surface 80 in a manner such that the upstream surface 80 would be illuminated directly by the light source for the purpose of decontamination. It is therefore a challenge to provide the upstream surface 80 of the filter assembly 32 with sufficient light such that effective sterilisation is performed.

The present invention is advantageous in that it provides a head wearable purifier that overcomes these challenges to allow for decontamination of the upstream surface of the filter assembly of the air purifier while the air purifier is in use (or simply when it is being worn by the user). In particular, the present invention is advantageous in that existing parts of the air purifier may be used to guide light from a source of violet light—positioned in a part of the air purifier where space constraints are not so severe—to the upstream surface of the filter assembly for the decontamination thereof, as described in greater detail below.

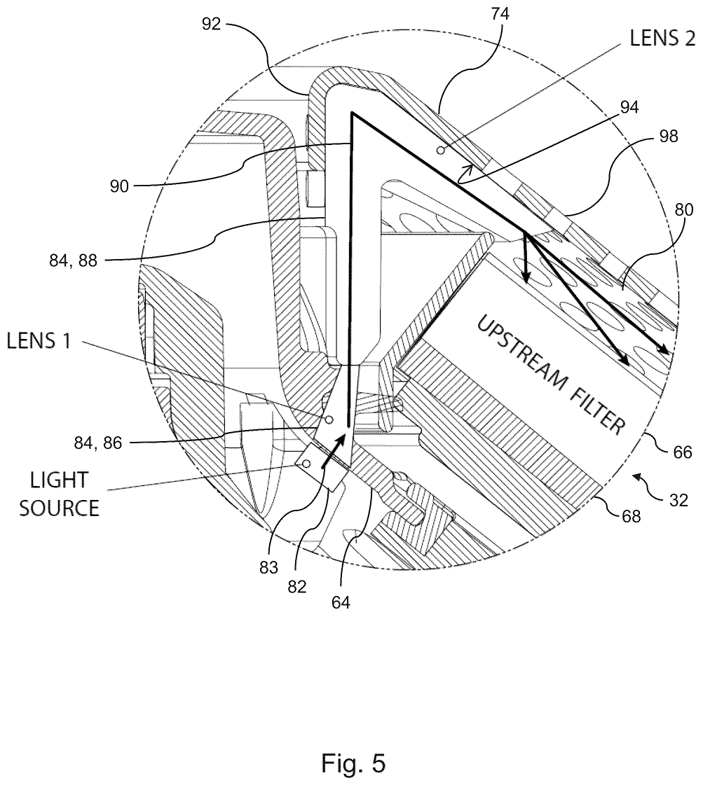

Returning to FIG. 2, and with additional reference to FIG. 5—which shows the section of the ear assembly 12 circled in FIG. 2—the air purifier 10 includes a light source 82 that is for emitting light in a violet portion of the visual spectrum. In the described example, the light source is in the form of a light emitting diode (LED) 82 that emits light with a wavelength of about 405 nm; however, different light sources could be used, and/or they could emit light at different wavelengths within the violet portion of the spectrum.

Violet light from the LED 82 is to be used to decontaminate at least the upstream surface 80 of the filter assembly 32. However, space constraints within the air purifier 10 mean that the LED 82 cannot be positioned such that it can emit light directly towards the upstream surface 80. Instead, the LED 82 is positioned elsewhere within the body of the purifier 10—in particular, in a part of the purifier 10 where the space constraints are not so severe—and its emitted light is guided by some means towards the filter assembly upstream surface 80.

In order that violet light emitted by the LED 82 can be used to sterilise the upstream surface 80 of the filter assembly 32, the air purifier 10 is provided with a light guide 84 arranged to guide the light emitted from the light source 82 to the upstream surface 80 (or upstream, filter 66) for the decontamination thereof. In the described example, the light guide is in the form of light piping means 84—i.e. one or more light pipes or light lens—arranged to guide light emitted from the violet light source 82 through the light piping means 84 to the upstream surface 80. In this way, the light guide 84 needs to be positioned relative to the light source 82 and to the upstream surface 80 of the filter assembly 32 such that the light guide 84 can receive light emitted by the light source 82 and guide or direct that light towards the upstream surface 80. The particular shape or configuration of the light guide 82 will therefore depend on the relative positions and orientations of the light source 82 and the upstream surface 80 of the filter assembly 32 in the air purifier 10. The light piping may formed from a plastic material, e.g. acrylic, polycarbonate or polypropylene, or may include the use of polylactic acid

One option is to position the light source 82 adjacent to one of the structural components forming the body of the ear assembly 12 of the air purifier 10. The light source—in this case, an LED 82— may then be fixed or attached to the structural component. In the described example, the LED 82 is attached to the filter frame 64. In particular, because of space constraints the LED 82 may be attached to a side of the filter frame 64 that is opposite to the filter assembly 32.

In the described example, the LED 82 is oriented to shine light towards the filter frame 64, as indicated the arrow 83 in FIG. 5. In the described example, the light guide 84 includes two light pipes or lens 86, 88 to guide the light emitted from the light source 82 to the upstream surface 80. Such light pipes, e.g. clear tubes, are designed to carry light relatively short distances with high efficiency, and can be used to bend light around corners and in relatively tight spaces with minimal loss of light intensity. By directing the emitted light through light piping that extends through one of the components, such as the filter frame 64, a simple and compact solution is provided to guide the light to the desired location.

A first one of the light lens 86 is arranged adjacent to the light source 82 so that it receives light emitted therefrom. In particular, the first lens 86 extends through and beyond the filter frame 64 to guide the light from the side on which the LED 82 is disposed to the opposite side of the filter frame 64 (where the filter assembly 32 is positioned). The first lens 86 may be attached to the filter frame 64 to fix it in place. In some examples, the filter frame 64 itself may define part of the light guide for guiding the light therethrough.

In the described example, a second one of the light lens 88 is arranged adjacent to the first light lens 86 so that it receives the light emitted therefrom (which in turn has guided the light from the LED 82). In particular, the second lens 88 guides light from within the ear assembly 12—specifically, from a side of the filter assembly 32 opposite to, or different from, the upstream surface or side 80—to the upstream surface 80. In this way, the second lens 88 includes a corner to change the direction of the guided light in a desired manner to direct the light around the filter assembly 32 to its upstream surface 80.

As illustrated in FIG. 5, the second lens 88 may be arranged to emit the guided light adjacent to the upstream surface 80 and, in particular, in a direction substantially parallel to the upstream surface 80. This may help to ensure that a greater amount of the upstream surface 80 is illuminated with the violet light. Also, the second lens 88 may be arranged to emit or disperse the violet light in a number of different directions across or along the upstream surface 80, again to increase the coverage of the violet light to different parts or areas of the upstream surface 80. The light guide 84 may therefore guide the light emitted by the LED 82 generally in the direction of the arrow 90.

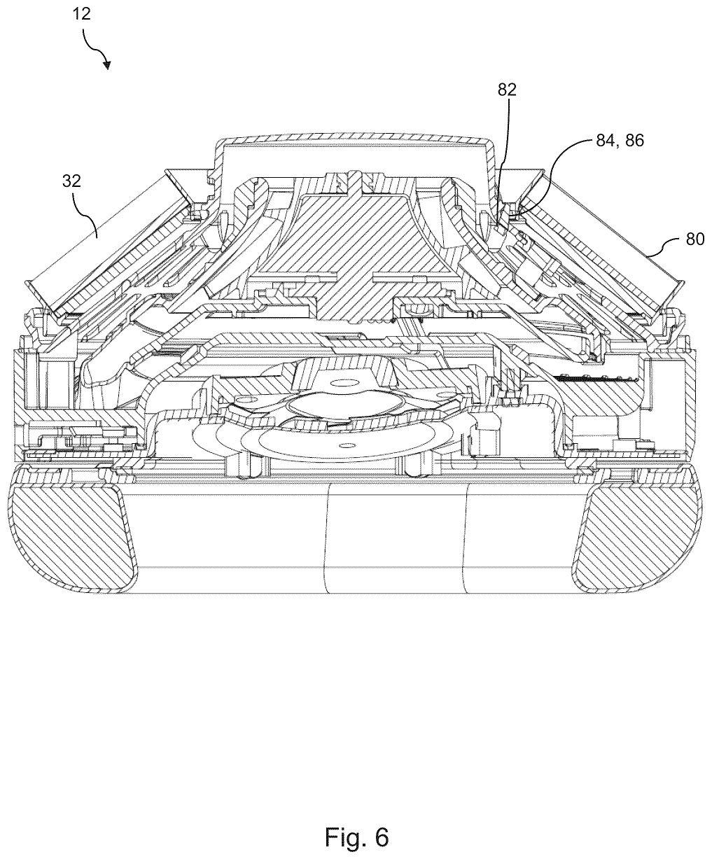

Conveniently, the second lens 88 may be attached to (or part of) the outer cover or shroud 74 of the air purifier 10. As illustrated in FIG. 5, the outer cover 74 may include a lip portion 92 that engages with, or clips onto, another structural component of the ear assembly 12, e.g. the filter frame 64, to secure the outer cover 74 in place to protect the filter assembly 32. For instance, the lip portion 92 may be at an inner side of the filter assembly 32. The second lens 88 may then abut an inner side 94 of the outer cover 74, and the lip portion 92 of the outer cover 74 may also define or shape a corner of the second lens 88 to direct light to the upstream surface 80. As noted above, the outer cover 74 is detachable or removable from the rest of the ear assembly 12. FIG. 6 shows a sectional view of the ear assembly 12 as in FIG. 2, but (unlike in FIG. 2) with the outer cover 74 removed. With reference to FIG. 6, in examples in which the second lens 88 is attached to the outer cover 74, the second lens 88 is also removed when the outer cover 74 is detached. However, as the first lens 86 and the violet light source 82 are fixed to the filter frame 64 then these parts 82, 86 remain in place when the outer cover 74 is removed.

When the outer cover 74 is fixed in place, the violet light (from the LED 82) that exits through the second lens 88 may be directed into a gap or space defined between the filter assembly 32 and the outer cover 74. This light irradiates (at least) the filter assembly upstream surface or side 80 in order to provide a decontaminating effect for components of the filter assembly 32.

By directing the light exiting the light guide into a space defined between the filter assembly and outer cover, more of the violet light irradiates the filter frame upstream surface 80 to increase the effectiveness of the filter decontamination. This is because the outer cover 74 helps in preventing light exiting the filter frame 64 from dissipating in various directions. For instance, the inner surface 94 of the outer cover 74—i.e. the surface facing the filter assembly 32—may have a reflective coating to increase the amount of light reflected back to the filter surface, which helps to ensure that most or all of the filter surface is irradiated with sufficient light to achieve the desired levels of decontamination or sterilisation.

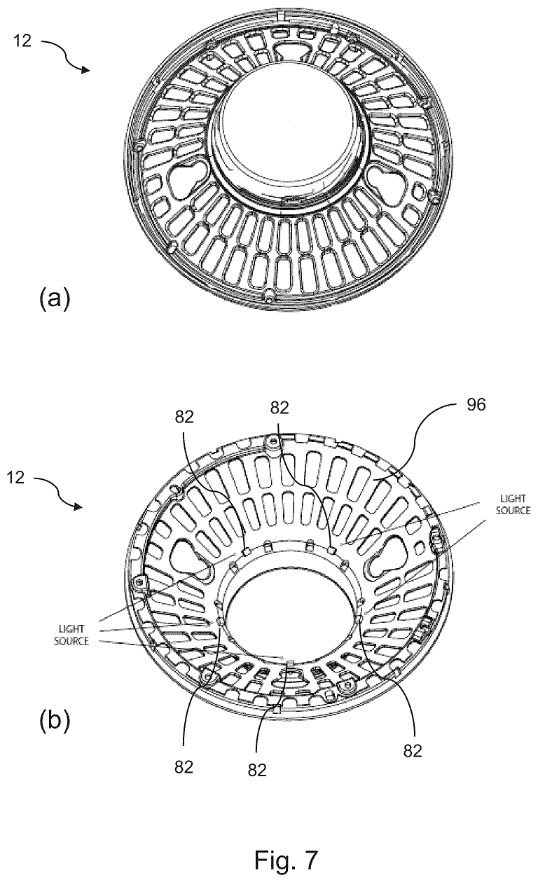

Although only one LED 82 is shown in the sectional views of FIGS. 2, 5 and 6, with additional reference to FIG. 7 the air purifier 10 of the described example includes a plurality of the LEDs 82. In particular, as illustrated in FIGS. 7(a) and 7(b), a number of the LEDs 82 are disposed around (or adjacent to) the circumference of the filter frame 64, specifically fixed to an underside 96 of the filter frame 64 (opposite to a side facing the filter assembly 32) as shown in FIG. 7(b). In examples in which the filter assembly 32 is generally conical or frustoconical, provision of the light source 82 around the circumference or perimeter of the ear assembly 12 allows for irradiation of the filter assembly upstream surface 80 around its full circumference. Although FIG. 7 shows five LEDs 82, any appropriate number of the LEDs may be disposed around the filter frame to achieve the required amount of radiation balanced against space, cost and weight constraints of including the LEDs in the ear assembly 12.

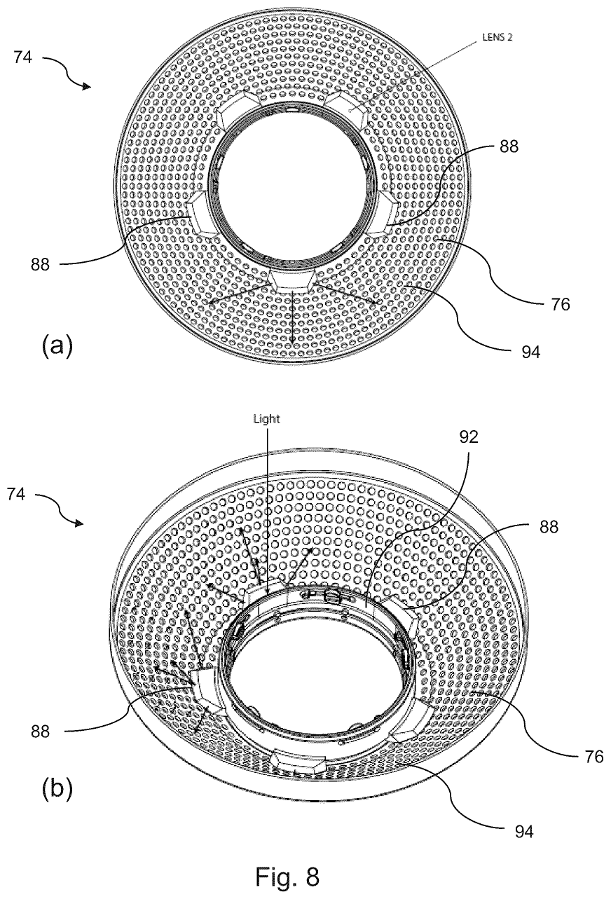

FIGS. 8(a) and 8(b) show perspective views of the underside of the outer cover 74. In particular, it is shown that there are a plurality second lenses 88 disposed around the circumference of the lip portion 92 of the outer cover 74, where each of the second lenses 88 corresponds to one of the plurality of LEDs 82. Note that a plurality of first lenses 86 are also included, corresponding to the respective LEDs 82 and second lenses 88. The apertures 76 in the outer cover 74 illustrate where air is drawn into the filter assembly 32. The arrows in FIGS. 8(a) and 8(b) illustrate how the second lenses 88 emit the violet light from the LEDs 88 in different directions in the space or gap defined between the filter assembly inner surface 80 and the inner surface 94 of the outer cover 74 (when the outer cover 74 is attached to the rest of the ear assembly 12). The arrows also indicate how the inner surface 94 of the outer cover 74 reflects the light back towards the filter assembly outer surface 80 to increase the amount of light that irradiates the outer surface 80, thereby improving the efficacy of the decontamination.

The sterilisation or decontamination process may be instigated in any appropriate manner. For instance, the head wearable air purifier 10 may include a switch (not shown) for switching the light source 82 between an on state, in which the light source 82 emits light, and an off state, in which the light source 82 does not emit light. The switch could for example be user operable. Alternatively, the light source 82 could be arranged to switch on when the motor-driven impeller is operational, which itself could be user operated. Further alternatively, the light source 82 could be arranged to switch on when the nozzle 16 is attached to the ear assemblies 12, or fixed to a particular (rotational) position relative to the ear assemblies 12, indicative that the air purifier 10 is in use. The light emitted from the light source 82 can be in any suitable manner, e.g. continuous, periodic, pulsed, etc.

Many modifications may be made to the examples described herein without departing from the scope of the appended claims.

Although an example is described in which the light guide includes two light lens, it will be appreciated that the light guide may contain a greater or fewer number of light lens to direct light in a desired manner.

In the described example, light piping in the form of the first lens 86 is used to guide the light emitted by the light source 82 from one side of the filter frame 64 to the other. In different examples, however, the filter frame 64—or any other one of the structural components in a light path between the light source 82 and the (upstream surface 80 of the) filter assembly 32—may be formed from a material through which light may pass. For instance, the filter frame 64 may be formed from a (transparent) plastics material such as acrylic. In such examples, the light source 82 may shine light directly through the filter frame 64, and this light may then be received by the second lens 88, for instance, to be directed to the upstream surface 80.

In the described example, the light source (in the form of one or more LEDs 82) is disposed on, or attached to, the filter frame 64. In different examples, the light source may additionally or alternatively be disposed at a different position in the ear assembly 12, where there is space to do so. For instance, the light source may be disposed on, or attached to, a different structural component of the ear assembly 12, such as the housing 18. The emitted light may then be guided from the light source to the filer assembly upstream surface by a suitably shaped and oriented light guide, possibly in combination with the emitted light being shone directly through one or more of the structural components formed from a transparent material such as acrylic.

In the above-described example, the light piping may extend through structural components such as the filter frame. In different examples, at least part of the light piping may be defined by one or more structural components such as the filter frame. In such examples, the structural component may be formed from an optically clear material and the light is guided through the structural component (to the filter assembly upstream surface).

In the above-described example, a light guide in the form of light piping is used to guide light emitted from the light source to the filter surface. In different examples, the light guide may additionally or alternatively take a different form. For instance, one or more reflective surfaces within the air purifier may be used to appropriately guide or direct the emitted light from the light source to the filter surface. Therefore, in the broadest sense of the invention, the air purifier includes a light guide arranged to guide light emitted from one or more light sources to illuminate the filter assembly upstream surface in order to perform desired sterilisation or decontamination.

In the above-described example, light emitted from the light source is guided to the upstream surface of the filter assembly. This is because irradiation of this surface with violet light maximises the sterilisation effect on the filter and has the greatest impact on ensuring continuing effective operation of the filter. However, in some examples additional parts of the filter may be irradiated with violet light from the provided light source. For instance, a light guide may be used to direct light from the light source to the downstream surface of the filter assembly, optionally using light piping through the filter frame.

Note that light irradiation of the filter assembly upstream surface may still be performed in the absence of the outer cover in some examples. For instance, the light guide may not be attached to the outer cover and may be directed towards (rather than parallel to) the filter surface so that a greater amount of the emitted light reaches the filter surface.

1. A head wearable air purifier, comprising:
a filter assembly;
a motor-driven impeller for creating an airflow through the filter assembly to obtain a filtered airflow downstream of the filter assembly;
at least one light source for emitting light in a violet portion of the visual spectrum; and,
a light guide arranged to guide light emitted from the at least one light source to illuminate at least part of an upstream surface of the filter assembly for the decontamination thereof.

2. The head wearable air purifier according to claim 1, wherein the light guide comprises light piping means arranged to guide light emitted from the at least one light source through the light piping means to the upstream surface of the filter assembly.

3. The head wearable air purifier according to claim 2, wherein the light piping means comprises at least one corner arranged to cause a change of direction of light being guided through the light piping means.

4. The head wearable air purifier according to claim 2, wherein at least part of the light piping means extends through, or is defined by, at least one structural component of the head wearable air purifier.

5. The head wearable air purifier according to claim 4, wherein the at least one structural component includes a filter frame that is arranged to support the filter assembly.

6. The head wearable air purifier according to claim 1, wherein the light guide is arranged to emit light in a direction substantially parallel to the upstream surface of the filter assembly.

7. The head wearable air purifier according to claim 1, wherein the light guide is arranged to emit light in a plurality of directions along the upstream surface of the filter assembly.

8. The head wearable air purifier according to claim 1, the head wearable air purifier comprising a filter shroud for covering the upstream surface of the filter assembly, wherein light from the at least one light source illuminates a gap defined between the filter shroud and the upstream surface of the filter assembly.

9. The head wearable air purifier according to claim 8, wherein an inner surface of the filter shroud, adjacent to the upstream surface of the filter assembly, comprises a light reflective coating, or is made from a light reflective material.

10. The head wearable air purifier according to claim 8, wherein a part of the light guide from which light, from the at least one light source, is emitted is positioned between the filter shroud and the upstream surface of the filter assembly.

11. The head wearable air purifier according to claim 8, wherein the filter shroud comprises a lip portion for fixing the filter shroud in place to cover the upstream surface of the filter assembly.

12. The head wearable air purifier according to claim 8, wherein at least part of the light guide is attached to the filter shroud.

13. The head wearable air purifier according to claim 1, wherein the at least one light source is arranged at a side of the filter assembly opposite to the upstream surface of the filter assembly.

14. The head wearable air purifier according to claim 1, wherein the at least one light source is attached to, or disposed adjacent to, at least one structural component of the head wearable air purifier.

15. The head wearable air purifier according to claim 14, wherein the at least one structural component is between the at least one light source and the light guide.

16. The head wearable air purifier according to claim 15, wherein the at least one structural component is formed from a material that allows light emitted from the at least one light source to pass therethrough.

17. The head wearable air purifier according to claim 16, wherein the material is a plastic material, optionally wherein the plastic material is acrylic, polycarbonate or polypropylene, or includes the use of polylactic acid.

18. The head wearable air purifier according to claim 14, wherein the at least one structural component includes a filter frame that is arranged to support the filter assembly.

19. The head wearable air purifier according to claim 1, wherein the at least one light source is in the form of one or more LEDs.

20. The head wearable air purifier according to claim 1, wherein the at least one light source is configured for emitting light with a wavelength of about 405 nm.

21. The head wearable air purifier according to claim 1, the head wearable air purifier comprising switch means for switching the at least one light source between an on state, in which the light source emits light, and an off state, in which the light source does not emit light, optionally wherein the switch means is user operable.

22. The head wearable air purifier according to claim 1, the head wearable air purifier comprising an ear assembly arranged to be worn over an ear of a user, wherein the ear assembly includes the filter assembly, the motor-driven impeller and the at least one light source;
optionally wherein the ear assembly is a first ear assembly arranged to be worn over a first ear of the user, the head wearable air purifier comprising a second ear assembly arranged to be worn over a second ear of a user, wherein the second ear assembly includes a second filter assembly, a second motor-driven impeller for creating an airflow through the second filter assembly to obtain a filtered airflow downstream of the second filter assembly, and at least one second light source for emitting light in a violet portion of the visual spectrum.

23. The head wearable air purifier according to claim 1, the head wearable air purifier including a nozzle for receiving the filtered air downstream of the filter assembly, and for directing the filtered air towards a user's mouth.
